(19) United States
(12) Patent Application Publication
(10) Pub. No.: US 2007/0197989 A1
(43) Pub. Date: Aug. 23, 2007

(54) Hygiene product with far infrared anions

(76) Inventor: Kuei-Min Chi, Taoyuan City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/651,045

(22) Filed: Jan. 9, 2007

(30) Foreign Application Priority Data
Feb. 23, 2006 (TW) 095203066
Feb. 23, 2006 (TW) 095203069

(51) Int. Cl. A61F 13/15

(52) U.S. Cl. 604/367; 604/385.01

(57) ABSTRACT

The present invention discloses a hygiene product with far infrared anions, and the hygiene product is made of an external leak-proof layer and a middle absorbent layer made of a water absorbent material, and an internal contact layer made of a water penetrating material. The fibers of the materials are attached with a constituent capable of releasing far infrared anions to achieve a sanitary anti-bacteria effect and a nursing care function.

### BACKGROUND OF THE INVENTION

1. Field of the Invention

The present invention relates to a hygiene product with far infrared anions, and more particularly to a hygiene product produced by soaking a liquid far infrared anion substance into fibers of an internal layer of the hygiene product by either a dipping method or a coating method, so as to achieve the sanitary and anti-bacterial effects and the nursing care function.

2. Description of the Related Art

Menstruation is one of the characteristics of females, and a woman's first menstruation generally occurs at an average age of 13 years. Menstruation usually causes inconvenience to the daily life of females or even gives rise to menstrual cramp, dizziness and unwellness, and thus various different hygiene products are developed for female's menstrual cycle. In addition to basic functions, the hygiene products also may provide good physiological effects for a woman's body, and that is an important issue to women.

Ancient Egyptian women fashioned a kind of throw-away tampon probably made of papyrus or other grass for absorbing menstrual blood; ancient Greek women wrapped gunny onto a small piece of wood and used it as a tampon or adopted a clean broken piece of cloth as a menstrual pad which was used repeatedly after washing and cleaning; and ancient Chinese women placed a white long cloth at the lower part of the body for absorbing menstrual blood and washed the white cloth by clean water with alum for repeated uses. All of the abovementioned primitive methods result in poor cleaning of tampons and menstrual pads, and thus can no longer meet user requirements of the present time.

As time goes by, a more sanitary tampon is introduced. Most tampons were manufactured by and imported from developing countries such as U.S.A. and Japan at early stage. Tampons at early time were provided for the basic requirement of absorbing menstrual blood only and they are mainly made of a cotton material, and thus the quality was not as good. However, improvements have been made, and imported tampons do not fit the local market. Therefore, Taiwan and Japan started exchanging technologies and manufactured tampons on their own. Besides the design and size of the cotton pad, manufacturers also design different size and types of tampons for daytime, nighttime, heavy-duty or light-duty use. Comfort and sanitation are taken into consideration for the improvement of exterior materials of a tampon. Since an old model come with a plastic layer may cause a reflux of menstrual blood or produce unpleasant smell after a short time of use, therefore materials such as PE and unwoven cloth are adopted to enhance the comfort of use. To make the use of tampons more convenient, a tampon with wings is developed for fixing the tampon and guarding a possible overflow of menstrual blood, and another revolutionary product of ultra-thin tampons is introduced to eliminate those inapplicable tampons from the market.

Most of the present tampons emphasize on the abovementioned basic functions of absorbability, lightness, thinness, and dryness to overcome user's uncomfortable feeling of menstrual cycle. Inventors and manufacturers further developed a tampon structure with a function of adjusting physiological conditions of the user, and brought another revolution to hygiene products, and most products of this type are accomplished by far infrared radioactive objects. Although far infrared gives beneficial effects to the physiological conditions of a user, yet conventional far infrared radioactive objects general include ceramic powder materials in the layers of a tampon, and these powders may cause permeations at both internal and external layers. Since the size of such powder is very small, the powder may fall out from the tampon or even enters into the user's vagina that may cause infection and unwellness, and thus the prior art requires further improvements.

In view of the foregoing shortcomings, the inventor of the present invention based on years of experience in the related industry to conduct extensive researches and experiments, and finally developed a hygiene product with far infrared anions to overcome the shortcomings of the prior art and achieve the sanitary and anti-bacterial effects and the nursing care function.

### SUMMARY OF THE INVENTION

It is a primary object of the invention to provide a hygiene product with far infrared anions, wherein a constituent attached to fibers of the hygiene product and capable of releasing far infrared anions is a super-fine solution with excellent permeability that can be permeated into the fibers or integrated with the fibers without creating any powder waste. Thus, the present invention can overcome the shortcoming of the prior art that the powder waste may fall off or infect a vagina, and achieves the nursing care with safe, anti-bacterial and deodorant effects.

In order to achieve the above-mentioned object, the hygiene product with far infrared anions in accordance with the invention includes:

a) an external leak-proof layer;

b) a middle absorbent layer made of a water absorbent material; and

c) an internal contact layer made of a water penetrating material with fibers absorbed with a constituent capable of releasing far infrared anions, the constituent consisting of a liquid far infrared anion substance attached in the fibers of the internal contact layer by either a dipping method or a coating method.

### BRIEF DESCRIPTION OF THE FIGURES

FIG. 1 is a schematic view of a structure of the present invention;

FIG. 2 is an enlarged view of Portion 2 depicted in FIG. 1;

FIG. 3 is a schematic view of a structure of a preferred embodiment of the present invention; and

FIG. 4 is a schematic view of a structure of another preferred embodiment of the present invention.

### DETAILED DESCRIPTION OF THE PREFERRED EMBODIMENT

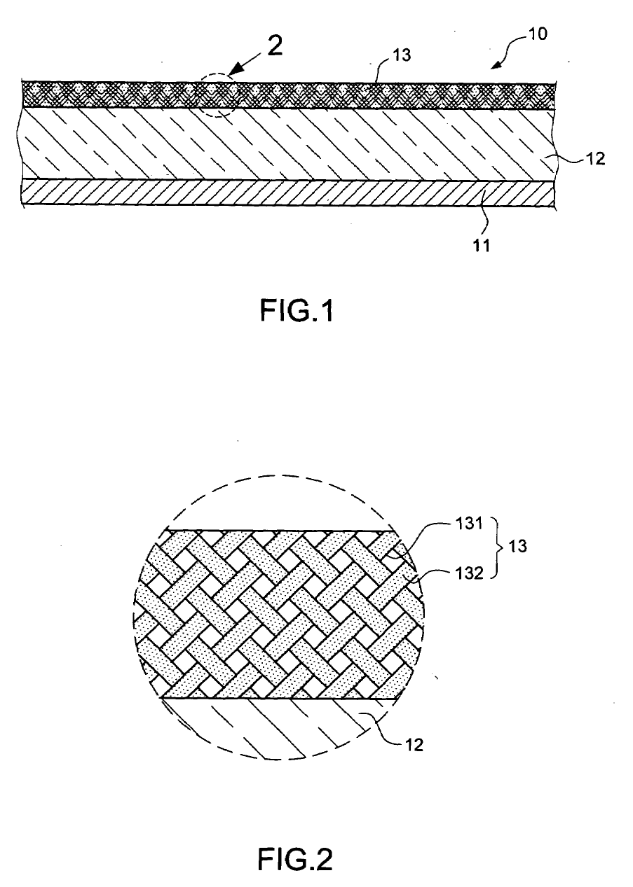

Referring to FIG. 1 for a hygiene product 10 with far infrared anions, the hygiene product 10 comprises an external leak-proof layer 11, a middle absorbent layer 12, and an internal contact layer 13.

The middle absorbent layer 12 is made of a water absorbent material for absorbing menstrual blood and preventing the menstrual blood from leaking and refluxing.

Referring to FIG. 2, the internal contact layer 13 is made of a water penetrating material, and the internal contact layer 13 is made of a material selected from the group consisting of unwoven cloth, cotton cloth, woven cloth, paper, and their equivalents. Further, a constituent 132 capable of releasing far infrared anions is attached to fibers 131 of the material, and the constituent 132 of far infrared anions is attached onto the fibers 131 of the internal contact layer 13 by a dipping method or a coating method. The constituent 132 of far infrared anions is a super fine solution with excellent permeability, which can be soaked into the fibers 131 and combined integrally with the fibers 131 without any leak of powder waste.

The constituent 132 soaked into the fibers 131 can be produced much easier and it also comes with a good durability for overcoming the cost for the loss of ceramic powder and provides all of the deodorant, anti-bacteria, dry and breathable, and blood circulating effects at a time.

Therefore, the constituent 132 of far infrared anions adopted in the present invention is a super fine far infrared anion liquid with excellent permeability, and the far infrared anion liquid is soaked into the fibers 131 and fully absorbed by the fibers 131 at the internal contact layer 13 of a hygiene product worn by a female user. In addition to the anti-bacteria and deodorant effects, the present invention also can release far infrared to achieve the nursing care effect.

The present invention adds the effect of the constituent 132 of far infrared anions, not only substituting the ceramic powder, but also enhancing the sanitary condition.

Further, the constituent 132 of far infrared anions is applied to a hygiene product for maximizing the deodorant effect to remove a stinky smell after the hygiene product has been used for a short period of time, as well as suppressing the reproduction and growth of bacteria, so as to assure a safety, sanitary and clean use of tampons for female users.

With the constituent 132 of far infrared anions, the lower body of a user can be moved freely for the effects of a better blood circulation and a more stable internal secretion.

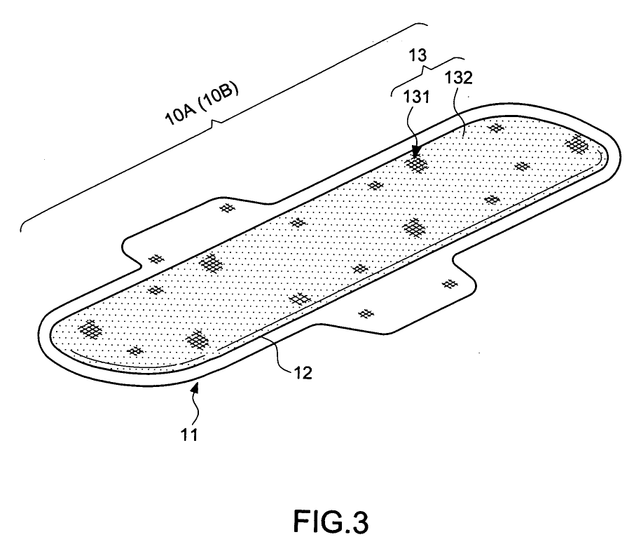

Referring to FIG. 3 for a schematic view of a structure of a preferred embodiment of the present invention, the hygiene product with far infrared anions 10 includes but not limited to a tampon 10A or a sanitary pad 10B. Regardless of tampon 10A or sanitary pad 10B, the fibers 131 of the internal contact layer 13 are soaked with the constituent 132 of far infrared anions for maximizing the anti-bacteria and deodorant effects.

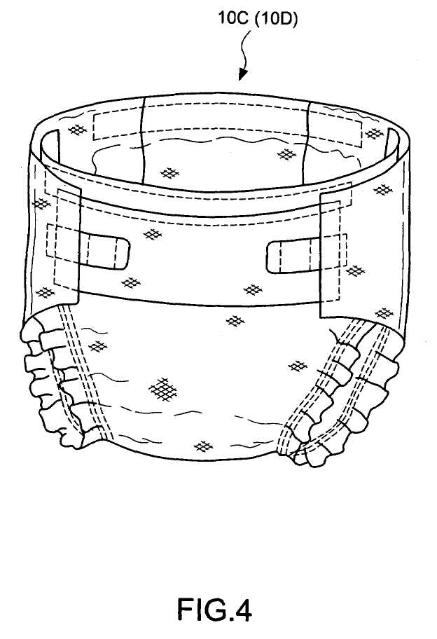

Referring to FIG. 4 for a schematic view of a structure of another preferred embodiment of the present invention, the technical characteristics of the present invention can be applied to a diaper 10C or a diaper cover 10D, in addition to the application of tampons for female users.

With the foregoing technical measures and extensive researches and experiments, the present invention definitely can achieve the expected effects and overcome the foregoing shortcomings of the prior art. The invention gives the deodorant and anti-bacteria effects and enhances the hygiene and nursing care functions.

Many changes and modifications in the above-described embodiments of the invention can, of course, be carried out without departing from the scope thereof. Accordingly, to promote the progress in science and the useful arts, the invention is disclosed and is intended to be limited only by the scope of the appended claims.

What is claimed is:

1. A hygiene product with far infrared anions, comprising:
a) an external leak-proof layer;
b) a middle absorbent layer made of a water absorbent material; and
c) an internal contact layer made of a water penetrating material with fibers absorbed with a constituent capable of releasing far infrared anions, the constituent consisting of a liquid far infrared anion substance attached in the fibers of the internal contact layer by either a dipping method or a coating method.

2. The hygiene product with far infrared anions as recited in 1, wherein the internal contact layer is made of a material selected from the group consisting of unwoven cloth, cotton cloth, woven cloth and paper.

3. The hygiene product with far infrared anions as recited in 1, wherein the hygiene product includes a tampon.

4. The hygiene product with far infrared anions as recited in 1, wherein the hygiene product includes a sanitary pad.

5. The hygiene product with far infrared anions as recited in 1, wherein the hygiene product is in a form selected from the group consisting of a diaper and a diaper cover.
